(19) United States
(12) Patent Application Publication
(10) Pub. No.: US 2019/0002782 A1
(43) Pub. Date: Jan. 3, 2019

(54) 13C-NMR-BASED COMPOSITION OF HIGH QUALITY LUBE BASE OILS AND A METHOD TO ENABLE THEIR DESIGN AND PRODUCTION AND THEIR PERFORMANCE IN FINISHED LUBRICANTS

(71) Applicant: ExxonMobil Research and Engineering Company, Annandale, NJ (US)

(72) Inventors: Charles L. BAKER, JR., Thornton, PA (US); Liezhong GONG, Basking Ridge, NJ (US); Eugenio SANCHEZ, Pitman, NJ (US); Angela R. HORTON, Spring, TX (US); Debra A. SYSYN, Monroe, NJ (US); Richard C. DOUGHERTY, Moorestown, NJ (US)

(21) Appl. No.: 16/012,913

(22) Filed: Jun. 20, 2018

Related U.S. Application Data
(60) Provisional application No. 62/527,418, filed on Jun. 30, 2017.

(51) Int. Cl. C10M 105/04; C10M 101/02; G01N 24/08; G01N 33/30; G01N 11/00

(52) U.S. Cl. CPC C10M 105/04; C10M 101/02; G01N 24/085; C10M 2203/1006; G01N 11/00; C10M 2203/024; G01N 33/30

(57) ABSTRACT

A lubricant base oil is provided. The lubricant base oil has a low temperature property determined using a stepwise regression of carbon-13 nuclear magnetic resonance (NMR) spectroscopy peak values. A method of selecting candidate lubricant base oils, or mixtures thereof, having acceptable low temperature performance is also provided. An online method of blending a lubricant base oil and a finished lubricant are also provided.

### CROSS-REFERENCE TO RELATED APPLICATIONS

This application claims the benefit of U.S. Provisional Application No. 62/527,418, filed on Jun. 30, 2017, the entire contents of which are incorporated herein by reference.

### FIELD

The present disclosure relates generally to lubricating base oils, a process for selecting lubricating base oils, and lubricating oil compositions.

### BACKGROUND

In the field of lubricating oils, additives such as pour point depressants have conventionally been added to lubricating base oils, including highly refined mineral oils, to improve properties such as the low-temperature viscosity characteristics of the lubricating oils. Known methods for producing high-viscosity-index base oils include methods in which feed stock oils containing natural or synthetic normal paraffins are subjected to lubricating base oil refining by hydrocracking or hydro-isomerization.

The properties evaluated for low-temperature viscosity characteristics of lubricating base oils and lubricating oils are generally the pour point, cloud point and freezing point. Methods are also known for evaluating the low-temperature viscosity characteristics for lubricating base oils according to their normal paraffin or isoparaffin contents.

The purpose of using lubricating oils in internal combustion engines, gearboxes and other mechanical devices is to produce smoother functioning in such devices. Internal combustion engine lubricating oils (engine oils), in particular, must exhibit high performance under the high-performance, high-output and harsh operating conditions of internal combustion engines. Various additives such as anti-wear agents, metal-based detergents, ashless dispersants and antioxidants are therefore added to conventional engine oils to meet such performance demands.

Finished lubricant performance is significantly affected by base oil parameters and composition. As indicated, one of the key performance parameters for finished lubricants are the low temperature properties, i.e. the viscosities experienced in various shear environments for different product applications. These viscosities are often influenced by both the nature of the test and the relatively low concentration of waxy components in the formulation. In addition, many lubricants are being formulated with greatly different types of base stocks such as Group II and Group III and PAO, where the amount and nature of the residual wax can vary greatly.

Viscosity index (VI) and pour point are important lubricant and industrial oil qualities that are typically used as manufacturing specifications and/or product specifications for base oils. There is a need to rapidly (in hours) estimate VI and pour point using a small quantity (<1 ml) of a base oil sample and to provide guidance for design, selection, and optimization of processes, including lubricant production processes, and catalysts to produce group I, II, and II+, III, III+, IV and other related isoparaffinic base stocks with the desired isomeric structures for optimal VI and pour point.

As such, need exists for a method to define acceptable compositions for base stocks that satisfy a range of low temperature properties, the base stocks employing mixed base stock systems and individual base stocks. The method may define acceptable compositions that satisfy a range of products, yielding a range of products that may be quickly and easily qualified.

### SUMMARY

In one aspect, provided is a finished lubricant. The finished lubricant comprises a lubricant base oil having a low temperature property (LTP) determined using a data analytics/machine learning technique on carbon-13 nuclear magnetic resonance (NMR) spectroscopy peak values.

In some embodiments, the data analytics/machine learning technique comprises stepwise regression, Bayesian regression, LASSO/Ridge regression, random forest, support vector machine, deep learning techniques or the like.

In some embodiments, the data analytics/machine learning technique comprises stepwise regression, and the spectroscopy peak values used in the stepwise regression are significant at the 90 percent confidence level. In some embodiments, the spectroscopy peak values used in the stepwise regression are significant at the 95 percent confidence level.

In some embodiments, the finished lubricant is an industrial oil.

In some embodiments, the stepwise regression utilizes at least three spectroscopy peak values.

In some embodiments, the stepwise regression equation is a−b*P15+c*P17−d*P18+e*(P2+P4+P10)<LN (Scanning Brookfield Viscosity@−30° C.=30,000). In some embodiments, a=11.06; b=2.857; c=0.811; d=3.328 and e=2.966.

In some embodiments, the finished lubricant is an engine oil suitable for operating under high shear.

In some embodiments, the stepwise regression utilizes at least three spectroscopy peak values.

In some embodiments, the stepwise regression equation is a+b*P17+c*P118−d*P15+e*(P2+P4+P10)−f*(P1+P5)<LN (Cold Cranking Simulator Viscosity@−25° C.=7,000). In some embodiments, a=9.093; b=0.4957; c=2.842; d=1.850, e=2.094 and f=1.964.

In some embodiments, the low temperature property is Mini Rotary Viscometer viscosity, ASTM D4684.

In some embodiments, the stepwise regression equation is a−b*P18+c*(P2+P4)<LN (Mini Rotary Viscosity@−30° C.=40,000). In some embodiments, a=12.18; b=4.16; and c=3.24.

In a further aspect, provided is a method of selecting candidate lubricant base oils, or mixtures thereof, having acceptable low temperature performance. The method comprises evaluating a set of samples using carbon-13 nuclear magnetic resonance (NMR) spectroscopy, each of the samples having a low temperature property; performing a data analytics/machine learning technique on the carbon-13 NMR spectroscopy peak values obtained for the set of samples and their low temperature properties; selecting the carbon-13 NMR spectroscopy peak values found to be significant at the at least the 90% confidence level for the selected low temperature property; selecting a candidate lubricant base oil based upon the data analytics/machine learning technique.

In some embodiments, the lubricant base oil is used to formulate a high shear engine oil.

In some embodiments, the set of samples span isoparaffin-containing base oils such as Group II, III and IV base oils.

In a yet further aspect, provided is a lubricant base oil, the lubricant base oil having a low temperature property determined using a stepwise regression of carbon-13 nuclear magnetic resonance (NMR) spectroscopy peak values.

In some embodiments, the spectroscopy peak values used in the stepwise regression are significant at the 90 percent confidence level.

In some embodiments, the spectroscopy peak values used in the stepwise regression are significant at the 95 percent confidence level.

In a yet still further aspect, provided is an online method of blending a lubricant base oil, the method comprising evaluating a set of samples using carbon-13 nuclear magnetic resonance (NMR) spectroscopy, each of the samples having a low temperature property; performing a data analytics/machine learning technique on the carbon-13 NMR spectroscopy peak values obtained for the set of samples and their low temperature properties; selecting the carbon-13 NMR spectroscopy peak values found to be significant at the at least the 90% confidence level for the selected low temperature property; monitoring online the carbon-13 NMR spectroscopy peak values of a first lubricant base oil blending component; monitoring online the carbon-13 NMR spectroscopy peak values of at least a second lubricant base oil blending component; mathematically determining the optimal blend ratio of the first lubricant base oil blending component and the at least second lubricant base oil blending component; and blending the first lubricant base oil blending component and the at least second lubricant base oil blending component in accordance with the optimal blend ratio to form a lubricant base oil.

In some embodiments, it may be desirable to use a different viscosity base oil. In such embodiments, the functional equations may be made relative to a well-known standard, such as API Group IV base stocks, especially 4, 6 and 8 cSt PAO.

In some embodiments, a ratio of other suitable techniques relative to the different viscosity can be used. For example, for any of the low temperature property predictions, an equation of the following form could be used: Predicted LTP Viscosity (Baseoil)<1.2*Predicted LTP Viscosity (PAO), where the viscosity of the PAO is the appropriate viscosity of reference.

In some embodiments, the reference viscosity range of the PAO may extend from 2 to 150 cSt @ 100° C.

In some embodiments, the form of the equation may be more complex to comprehend expected non-linearities. An example may be: Predicted LTP Viscosity (Baseoil)<1.2*F (29 cSt/kV40)*Predicted LTP Viscosity (PAO), where F(argument) is a function that could be a linear form, or could be exponential, logarithmic or a power law.

### BRIEF DESCRIPTION OF THE DRAWINGS

The present disclosure is susceptible to various modifications and alternative forms, specific exemplary implementations thereof have been shown in the drawings and are herein described in detail. It should be understood, however, that the description herein of specific exemplary implementations is not intended to limit the disclosure to the particular forms disclosed herein. This disclosure is to cover all modifications and equivalents as defined by the appended claims. It should also be understood that the drawings are not necessarily to scale, emphasis instead being placed upon clearly illustrating principles of exemplary embodiments of the present invention. Moreover, certain dimensions may be exaggerated to help visually convey such principles. Further where considered appropriate, reference numerals may be repeated among the drawings to indicate corresponding or analogous elements. Moreover, two or more blocks or elements depicted as distinct or separate in the drawings may be combined into a single functional block or element. Similarly, a single block or element illustrated in the drawings may be implemented as multiple steps or by multiple elements in cooperation. The forms disclosed herein are illustrated by way of example, and not by way of limitation, in the figures of the accompanying drawings and in which like reference numerals refer to similar elements and in which:

FIG. 1 presents a 13C-NMR spectrum of a base oil sample with chemical shifts corresponding to the aliphatic isomeric structures.

FIG. 2 presents a VI parity plot predicted from 13C-NMR analysis using the equation: VI=163.21-23.94*P10+16.167*P17-36.9*P18-5.399*P24-61.46*LOG(KV100)+63.97*(P1+P5)−132.3*(P2+P4).

FIG. 3 presents a pour point parity plot predicted from 13C-NMR analysis using the equation: Pour Point (in degree C.)=−20.26−10.21 P15+2.999 P17.

FIG. 4 presents a Scanning Brookfield of industrial gear oil (high performance), wherein LN (Scanning Brookfield Visc@−30° C.)=11.06-2.857*P15+0.811*P17-3.328*P18+2.966*(P2+P4+P10).

FIG. 5 presents a 10W-40 PCMO engine oil CCS parity plot, wherein LN(CCS@−25° C.)=9.093+0.4957*P17-2.842*P18-1.850*P15+2.094*(P2+P4+P10)-1.964*(P1+P5).

FIG. 6 presents a 10W-40 PCMO engine oil MRV plot @ −30° C., wherein LN(MRV@−30° C.)=12.18-4.16*P18+3.24*(P2+P4).

FIG. 7 presents regression plots against individual NMR peaks, showing the relationships, according to the present disclosure.

### DETAILED DESCRIPTION

### Terminology

The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than the broadest meaning understood by skilled artisans, such a special or clarifying definition will be expressly set forth in the specification in a definitional manner that provides the special or clarifying definition for the term or phrase.

For example, the following discussion contains a non-exhaustive list of definitions of several specific terms used in this disclosure (other terms may be defined or clarified in a definitional manner elsewhere herein). These definitions are intended to clarify the meanings of the terms used herein. It is believed that the terms are used in a manner consistent with their ordinary meaning, but the definitions are nonetheless specified here for clarity.

A/an: The articles “a” and “an” as used herein mean one or more when applied to any feature in embodiments and implementations of the present invention described in the specification and claims. The use of “a” and “an” does not limit the meaning to a single feature unless such a limit is specifically stated. The term “a” or “an” entity refers to one or more of that entity. As such, the terms “a” (or “an”), “one or more” and “at least one” can be used interchangeably herein.

About: As used herein, “about” refers to a degree of deviation based on experimental error typical for the particular property identified. The latitude provided the term “about” will depend on the specific context and particular property and can be readily discerned by those skilled in the art. The term “about” is not intended to either expand or limit the degree of equivalents which may otherwise be afforded a particular value. Further, unless otherwise stated, the term “about” shall expressly include “exactly,” consistent with the discussion below regarding ranges and numerical data.

Above/below: In the following description of the representative embodiments of the invention, directional terms, such as “above”, “below”, “upper”, “lower”, etc., are used for convenience in referring to the accompanying drawings. In general, “above”, “upper”, “upward” and similar terms refer to a direction toward the earth's surface along a wellbore, and “below”, “lower”, “downward” and similar terms refer to a direction away from the earth's surface along the wellbore. Continuing with the example of relative directions in a wellbore, “upper” and “lower” may also refer to relative positions along the longitudinal dimension of a wellbore rather than relative to the surface, such as in describing both vertical and horizontal wells.

And/or: The term “and/or” placed between a first entity and a second entity means one of (1) the first entity, (2) the second entity, and (3) the first entity and the second entity. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements). As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of”.

Any: The adjective “any” means one, some, or all indiscriminately of whatever quantity.

At least: As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements). The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

Based on: “Based on” does not mean “based only on”, unless expressly specified otherwise. In other words, the phrase “based on” describes both “based only on,” “based at least on,” and “based at least in part on.”

Comprising: In the claims, as well as in the specification, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Determining: “Determining” encompasses a wide variety of actions and therefore “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

Embodiments: Reference throughout the specification to “one embodiment,” “an embodiment,” “some embodiments,” “one aspect,” “an aspect,” “some aspects,” “some implementations,” “one implementation,” “an implementation,” or similar construction means that a particular component, feature, structure, method, or characteristic described in connection with the embodiment, aspect, or implementation is included in at least one embodiment and/or implementation of the claimed subject matter. Thus, the appearance of the phrases “in one embodiment” or “in an embodiment” or “in some embodiments” (or “aspects” or “implementations”) in various places throughout the specification are not necessarily all referring to the same embodiment and/or implementation. Furthermore, the particular features, structures, methods, or characteristics may be combined in any suitable manner in one or more embodiments or implementations.

Exemplary: “Exemplary” is used exclusively herein to mean “serving as an example, instance, or illustration.” Any embodiment described herein as “exemplary” is not necessarily to be construed as preferred or advantageous over other embodiments.

May: Note that the word “may” is used throughout this application in a permissive sense (i.e., having the potential to, being able to), not a mandatory sense (i.e., must).

Operatively connected and/or coupled: Operatively connected and/or coupled means directly or indirectly connected for transmitting or conducting information, force, energy, or matter.

Optimizing: The terms “optimal,” “optimizing,” “optimize,” “optimality,” “optimization” (as well as derivatives and other forms of those terms and linguistically related words and phrases), as used herein, are not intended to be limiting in the sense of requiring the present invention to find the best solution or to make the best decision. Although a mathematically optimal solution may in fact arrive at the best of all mathematically available possibilities, real-world embodiments of optimization routines, methods, models, and processes may work towards such a goal without ever actually achieving perfection. Accordingly, one of ordinary skill in the art having benefit of the present disclosure will appreciate that these terms, in the context of the scope of the present invention, are more general. The terms may describe one or more of: 1) working towards a solution which may be the best available solution, a preferred solution, or a solution that offers a specific benefit within a range of constraints; 2) continually improving; 3) refining; 4) searching for a high point or a maximum for an objective; 5) processing to reduce a penalty function; 6) seeking to maximize one or more factors in light of competing and/or cooperative interests in maximizing, minimizing, or otherwise controlling one or more other factors, etc.

Order of steps: It should also be understood that, unless clearly indicated to the contrary, in any methods claimed herein that include more than one step or act, the order of the steps or acts of the method is not necessarily limited to the order in which the steps or acts of the method are recited.

Ranges: Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a range of about 1 to about 200 should be interpreted to include not only the explicitly recited limits of 1 and about 200, but also to include individual sizes such as 2, 3, 4, etc. and sub-ranges such as 10 to 50, 20 to 100, etc. Similarly, it should be understood that when numerical ranges are provided, such ranges are to be construed as providing literal support for claim limitations that only recite the lower value of the range as well as claims limitation that only recite the upper value of the range. For example, a disclosed numerical range of 10 to 100 provides literal support for a claim reciting “greater than 10” (with no upper bounds) and a claim reciting “less than 100” (with no lower bounds).

### Description

Specific forms will now be described further by way of example. While the following examples demonstrate certain forms of the subject matter disclosed herein, they are not to be interpreted as limiting the scope thereof, but rather as contributing to a complete description.

Disclosed herein are lubricant base oils and finished lubricants, the lubricant base oils and finished lubricants having a low temperature property determined using a data analytics/machine learning technique on carbon-13 nuclear magnetic resonance (NMR) spectroscopy peak values. The present disclosure also provides a method of selecting candidate lubricant base oils, or mixtures thereof, having acceptable low temperature performance. The method includes evaluating a set of samples using carbon-13 NMR spectroscopy, each of the samples having a low temperature property; performing a data analytics/machine learning technique on the carbon-13 NMR spectroscopy peak values obtained for the set of samples and their low temperature properties; selecting the carbon-13 NMR spectroscopy peak values found to be significant at the at least the 90% confidence level for the selected low temperature property; and selecting a candidate lubricant base oil based upon the data analytics/machine learning technique.

In some embodiments, the data analytics/machine learning technique comprises stepwise regression, Bayesian regression, LASSO/Ridge regression, random forest, support vector machine, deep learning techniques or the like. In some embodiments, the data analytics/machine learning technique comprises stepwise regression.

The viscosity index (VI) and pour point of a base oil is typically measured using a viscometer and a pour point test apparatus. This equipment requires a large sample size (˜50 ml), and does not yield good correlations between structure and VI and pour point.

Disclosed herein are methods which use carbon-13 nuclear magnetic resonance spectroscopy (NMR) to select candidate lubricant base oils and finished lubricants having suitable low temperature properties. Carbon-13 NMR is frequently used to study the structure of molecules, the interaction of various molecules, the kinetics or dynamics of molecules and the composition of mixtures of biological or synthetic solutions or composites. The size of the molecules analyzed can range from a small organic molecule or metabolite, to a mid-sized peptide or a natural product, all the way up to proteins of several tens of kDa in molecular weight.

A carbon-13 NMR spectrometer is composed of a magnet, a sample probe, a transmitter and receiver, and a computer for instrument control and display of results. Magnets used in NMR spectroscopy are predominantly superconducting solenoid systems, in which a wire coil is immersed in liquid helium to render it superconducting. An electric current is passed through the coil and produces a static magnetic field proportional to the size of the current. The magnet has an open bore that has shim coils to compensate for imperfections in the magnetic field and in the sample probe coils. Liquid samples with a deuterated solvent are placed inside this coil.

An array of high-power radiofrequency (RF) transmitter channels is also necessary to perturb the nuclear spin distribution from equilibrium through the use of strong RF pulses. A transmitter comprises an RF source, a phase modulator for determining pulse phases during an experiment, an amplitude controller and a solid-state amplifier capable of pulses of up to several hundred watts. An excitation pulse tilts the net nuclear magnetization away from its equilibrium orientation parallel to the magnetic field axis, although it continues to process around this axis. The precession induces a voltage in the probe coil that is tuned to the resonance frequency of the observed nucleus. One of the transmitters is part of a channel (known as a lock channel) that is dedicated to the detection of 2H nuclei in deuterated solvents and is used to stabilize the magnetic field and permit the adjustment of field homogeneity. For both the observe and lock channels a high fidelity preamplifier and receiver are used to amplify and detect the signal and then route it to an analog-to-digital converter (ADC) and a noise filter system before converting it to a stored data file on a computer as a free induction decay (FID) signal. Software on the computer performs a Fourier transformation of the FID signal to convert the time base-data into a frequency spectrum for interpretation. Suitable NMR systems having utility in the practice of the methods disclosed herein are available from JEOL USA, Inc., of Peabody, Mass., and other sources.

In performing the stepwise regression of carbon-13 NMR spectroscopy peak values, the relevant peaks may be quantified by integration and then normalized to the P3 peak. This, of course, is different than an overall integration across swaths of the NMR spectrum. As those skilled in the art would understand, using the P3 peak would count only the ends of molecules and that the molecules being studied for base stocks and finished lubes are more complex, some ending with rings etc. Of course, more traditional integration methods may have utility in the practice of the present disclosure.

As indicated, parameters important base stock and finished product performance include low temperature requirements as well as VI and pour point are important qualities and are typically used as manufacturing specs and/or product specs for various groups of base oils. Quantitative knowledge of the structure-property relationship and accurate prediction of VI and pour point can enable the proper design, selection, and optimization of processes and catalysts to produce group I, II, and II+ base oils with the desired isomeric structures for optimal VI and pour point.

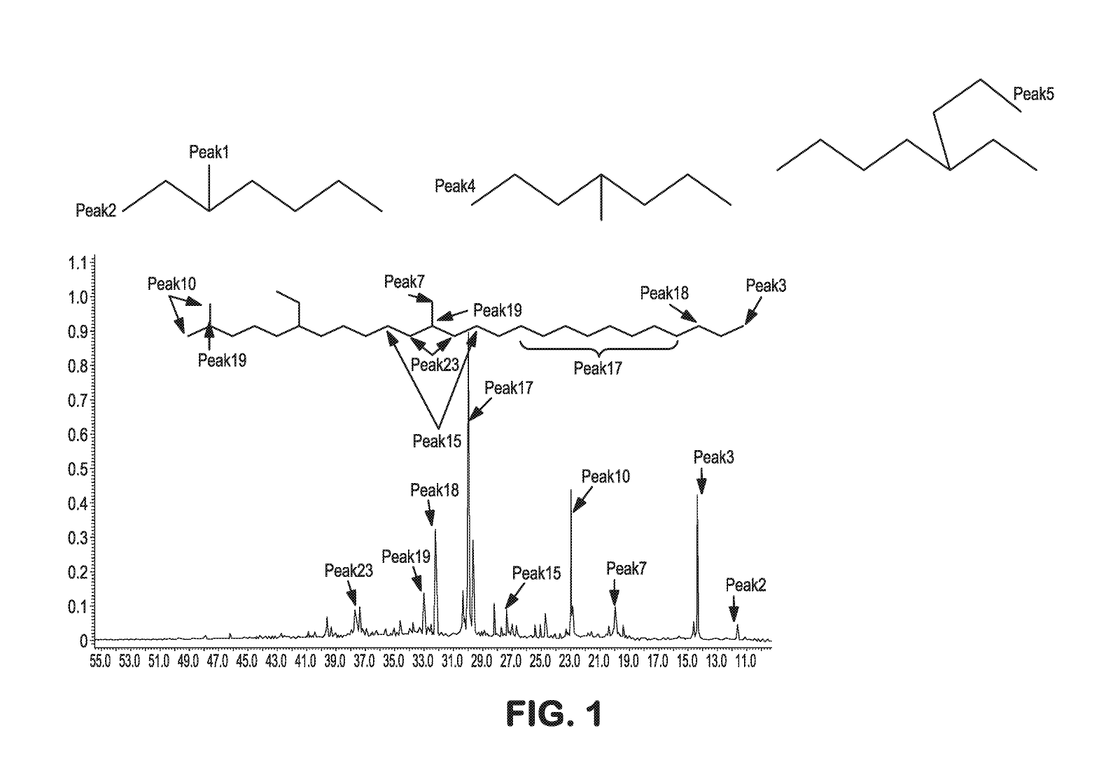

Referring now to FIG. 1, a 13C-NMR spectrum of a base oil sample with chemical shifts corresponding to the aliphatic isomeric structures. Over 70 samples were analyzed. The sample set comprised of dewaxed distillate, and group I, II and II+ base oils. The procedure used with the 13C-NMR spectrometer system was the procedure described in Fuel, 88, 2199-2206 (2009). The detailed isomeric structures shown in FIG. 1, and Table 1, below, were determined from 13C-NMR spectra obtained using the afore-mentioned procedure published in Fuel, 88, 2199-2206 (2009).

TABLE 1

| Stat ppm | End ppm | Description | Peak |
|---|---|---|---|
| 155.24 | 111.16 | Aromatics | P24 |
| 37.29 | 37.13 | Methylene groups in alpha carbon away | P23 |
| | | from methyl branch | |
| 32.94 | 32.69 | Tertiary carbon of from alpha methyl | P19 |
| | | branch | |
| 32.17 | 31.88 | Methylene groups in straight chain | P18 |
| | | (gamma) | |
| 30 | 29.67 | Methylene groups in straight chain | P17 |
| | | (epsilon carbon) | |
| 27.29 | 27.09 | Methylene groups in beta carbon away | P15 |
| | | from methyl branch | |
| 22.95 | 22.75 | Beta carbon of long chains | P11 |
| 22.75 | 22.46 | Methyl branching in Beta carbon (2-me) | P10 |
| 20.01 | 19.44 | Methyl branching more the 4 carbons from | P7 |
| | | terminal methyl | |
| 19.45 | 19.15 | Methyl branching on carbon 3 (3-me) | P6 |
| 14.66 | 14.53 | Methyl of pendant propyl branch | P5 |
| 14.53 | 14.37 | Methyl of terminal propyl group (4-me) | P4 |
| 14.32 | 13.90 1 | Methyl group at end of alkyl chain | P3 |
| 11.59 | 11.27 | Methyl of terminal ethyl group (3-me) | P2 |
| 11.14 | 10.69 | Methyl of pendant ethyl branch | P1 |

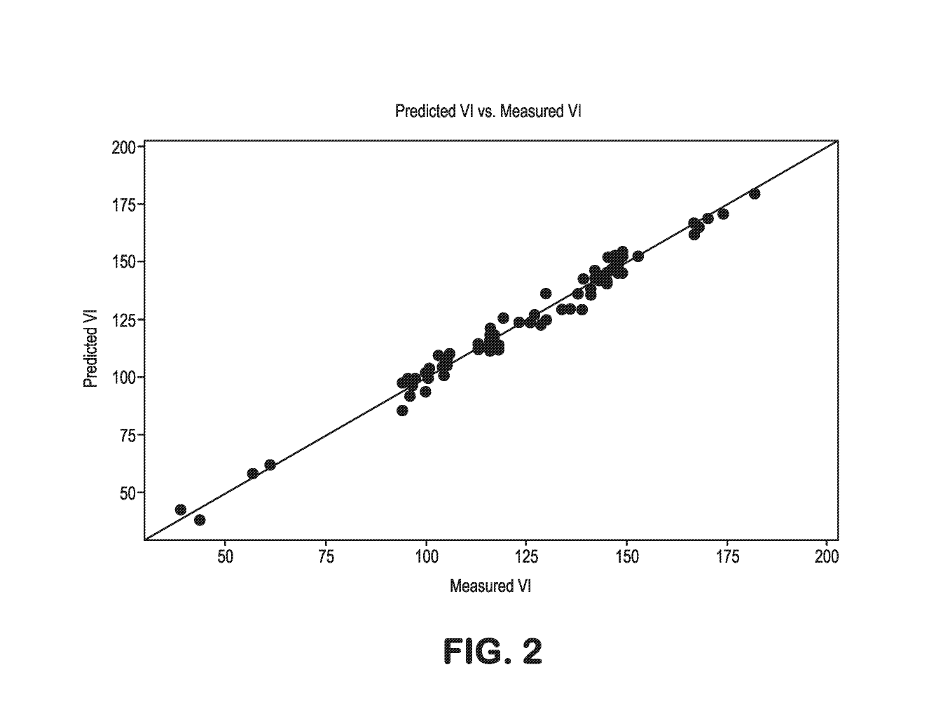

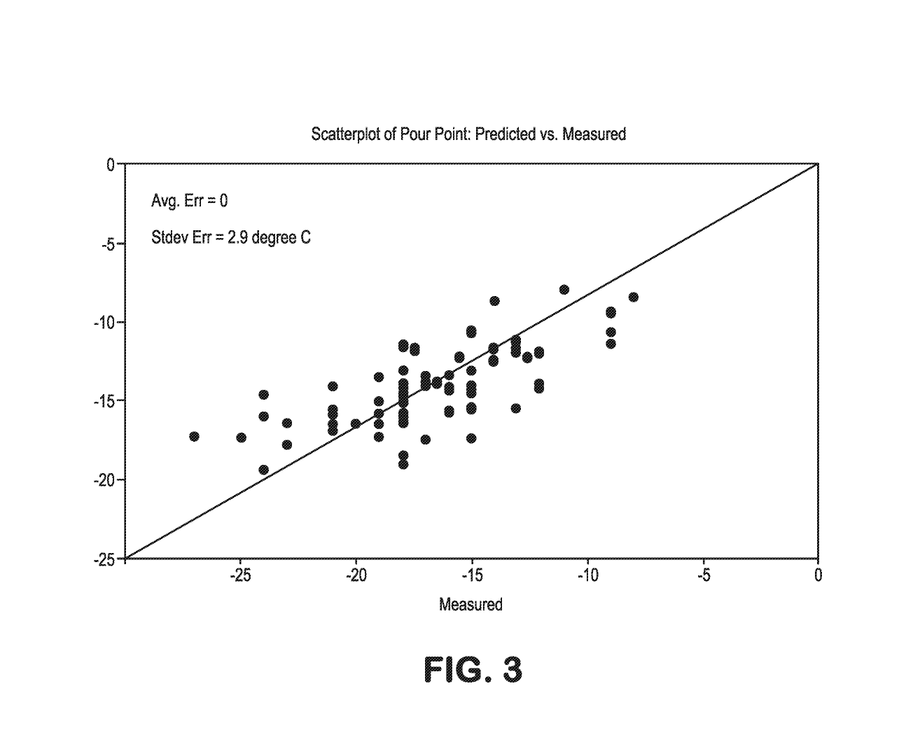

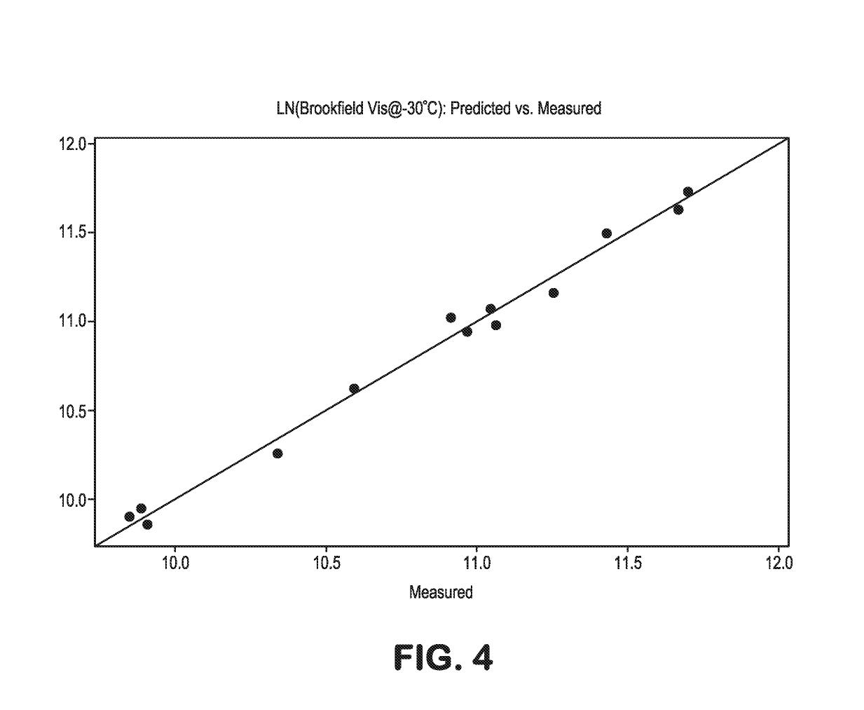

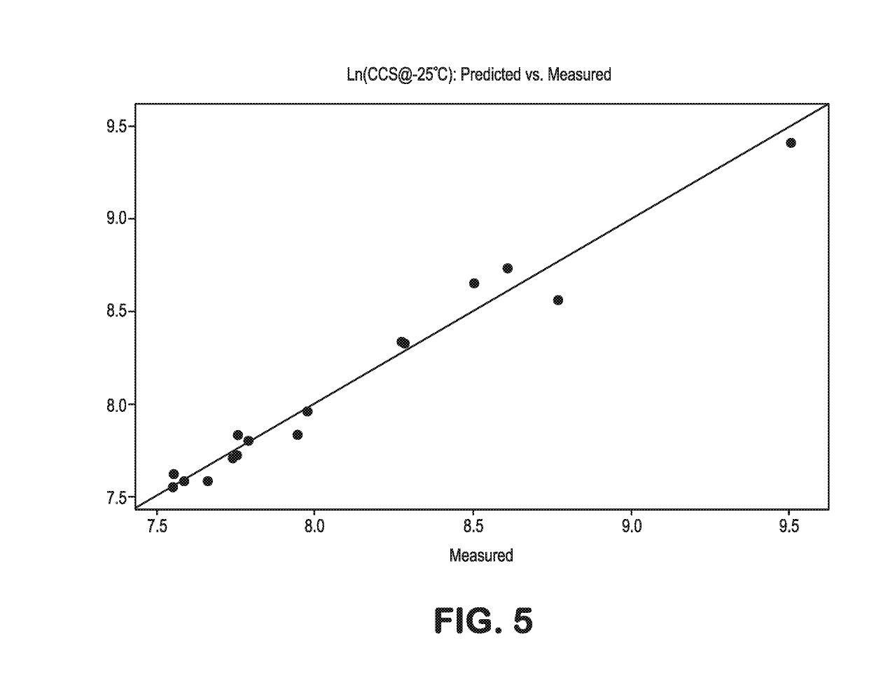

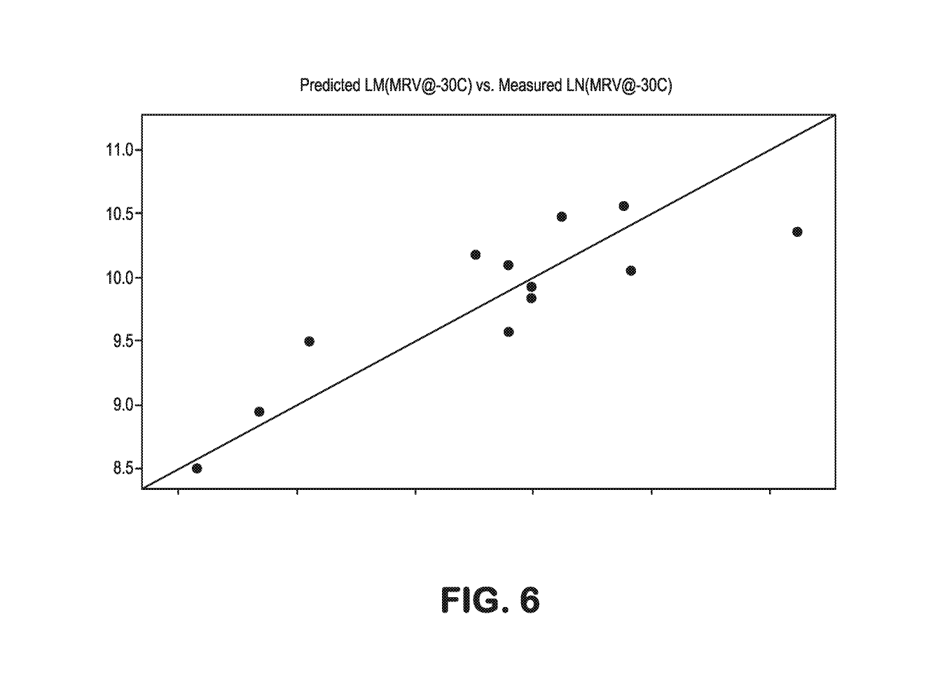

Using backward stepwise regression analysis, equations were established to predict the finished lube performance as well as VI and pour point using the 13C-NMR analysis data. As shown in FIGS. 2-6, the derived correlations demonstrated reasonable prediction accuracy and provided an excellent ability to estimate the VI and pour point of the samples. Examining the correlation equations in detail yielded the following principles governing the relationship between isomeric structure and base oil VI and pour point: terminal branches (P2+P4) reduce VI, increase CCS and Brookfield viscosity (ASTM D5133 including the entire curve); higher aromatics (Ar) reduce VI; higher viscosities (kv100) result in lower VI; undisrupted CH2 segments (peak 17, free carbons) increase VI, but also increase pour point, CCS, and Brookfield viscosity; more branches with uniform distribution (higher peak 15 and lower peak 17) decrease pour point, CCS, and Brookfield viscosity. In some embodiments, the spectroscopy peak values used in the stepwise regression are significant at the 90 percent confidence level. In some embodiments, the spectroscopy peak values used in the stepwise regression are significant at the 95 percent confidence level.

In some embodiments, the finished lubricant is an industrial oil. In some embodiments, the stepwise regression utilizes at least three spectroscopy peak values. In some embodiments, the stepwise regression equation is a−b*P15+c*P17−d*P18+e*(P2+P4+P10)<LN (Scanning Brookfield Viscosity@−30° C.=30,000). In some embodiments, a=11.06; b=2.857; c=0.811; d=3.328 and e=2.966.

In some embodiments, the finished lubricant is a high shear engine oil. In some embodiments, the stepwise regression utilizes at least three spectroscopy peak values. In some embodiments, the stepwise regression equation is a+b*P17+c*P118−d*P15+e*(P2+P4+P10)−f*(P1+P5)<LN (Cold Cranking Simulator Viscosity@−25° C.=7,000). In some embodiments, a=9.093; b=0.4957; c=2.842; d=1.850, e=2.094 and f=1.964.

In some embodiments, the low temperature property is Mini Rotary Viscometer viscosity. In some embodiments, the stepwise regression utilizes at least three spectroscopy peak values. In some embodiments, the stepwise regression equation is a−b*P18+c*(P2+P4)<LN (Mini Rotary Viscosity@−30° C.=40,000). In some embodiments, a=12.18; b=4.16; and c=3.24.

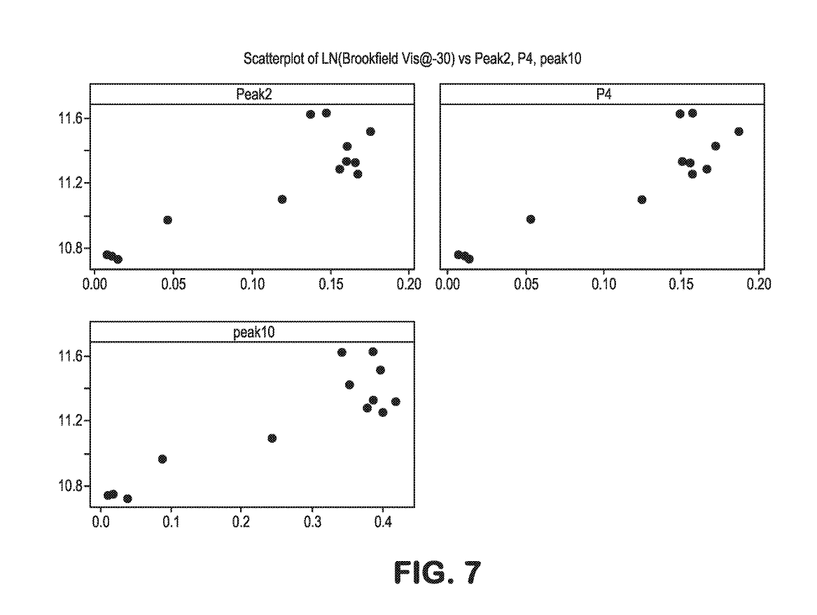

As shown in FIG. 7, while some results might conform to simpler equations, it has been found that the full range of NMR peaks and values are required for an effective correlation.

### Base Stock Processing

With respect to base stock processing, based on the principles derived herein, the ideal isomeric structures with high VI and low pour point/low scanning Brookfield viscosity/low CCS (ASTM D5293) and MRV (ASTM D4684) should possess the following characteristics: minimized terminal branches; a proper number of internal branches; and have short c carbons.

This structure-property relationship is useful for designing and/or making hydrocarbons with specific isomeric structures for optimal base oil performance. As an example, ZSM 48 tends to produce branches with random distribution and therefore is a desirable catalyst to make base stock, while ZSM 22 and ZSM 23 produce more terminal branches and are less ideal catalysts to make base stock.

### Base Stock Formulation

A variety of finished product blends were created encompassing both engine oils and industrial oils.

The base stocks used spanned commercial Gp II, III and IV base oils and had viscosities in the range of (4 to 11 cSt). For the blends, the base stocks were blended to a very narrow range of viscosities, i.e. 5.5 cSt @100° C. for the 10W-40 engine oil and about 29 cSt @ 40 C for the portion of the industrial oil. All other components for both engine oils and industrial oils were held constant. The engine oils used a range of base stocks) in a 10w-40 high performance PVL engine oil. The base stocks used were the majority of the base stocks in the formulation. The industrial oils used a range of base stocks with viscosity grades that were designed for very high performance using only a modest amount of the various base oils.

Based upon the composition, a series of base stocks were made or defined to meet demanding low temperature requirements.

As an example, an NMR spectra fulfilling the VI of a Group II (80 to 120) as well as the Scanning Brookfield and the MRV requirements in the industrial oil and engine oil respectively are as shown in Table 2, below.

TABLE 2

| | | | | | | | | | Predicted | | | |
|---|---|---|---|---|---|---|---|---|---|---|---|---|
| | | | | | | | | | Brookfield | Predicted | Predicted | Predicted |
| P10 | P15 | P17 | P18 | P24 | P1 + P5 | P2 + P4 | P2 + P4 + P10 | kv 100 C. | Visc@−30° C. | CCS@−25° C. | MRV@−30° C. | VI |
| 0.4 | 1.2 | 3.8 | 1.1 | 0 | 0.2 | 0.2 | 0.6 | 5.3 | 6851 | 661 | 3835 | 116 |
| 0.2 | 1.5 | 3.5 | 0.8 | 0 | 0.3 | 0.3 | 0.5 | 5.3 | 4599 | 511 | 18472 | 120 |
| 0.011 | 0.005 | 1.3 | 0.6 | 0 | 0.032 | 0.013 | 0.024 | 5.3 | 26223 | 3012 | 16749 | 118 |
| 0.5 | 1.7 | 5 | 0.9 | 0 | 0.3 | 0.5 | 1 | 5.3 | 27692 | 1593 | 23295 | 107 |
| 0.4 | 2.1 | 6.9 | 1.1 | 0 | 0.3 | 0.6 | 1 | 5.3 | 21192 | 1104 | 14017 | 120 |
| 0.3 | 0.5 | 2 | 1.1 | 0 | 0.3 | 0.3 | 0.6 | 5.3 | 11758 | 813 | 5303 | 83 |

As indicated above, disclosed herein is a method of selecting candidate lubricant base oils, or mixtures thereof, having acceptable low temperature performance. The method includes evaluating a set of samples using carbon-13 nuclear magnetic resonance (NMR) spectroscopy, each of the samples having a low temperature property; performing a stepwise regression of the carbon-13 NMR spectroscopy peak values obtained for the set of samples and their low temperature properties; selecting the carbon-13 NMR spectroscopy peak values found to be significant at the at least the 90% confidence level for the selected low temperature property; and selecting a candidate lubricant base oil based upon the regression equation. In some embodiments, the spectroscopy peak values used in the stepwise regression are significant at the 95 percent confidence level.

In some embodiments, the lubricant base oil is used to formulate an industrial oil. In some embodiments, the stepwise regression utilizes at least three spectroscopy peak values. In some embodiments, the stepwise regression equation is a−b*P15+c*P17−d*P18+e*(P2+P4+P10)<LN (Scanning Brookfield Viscosity@−30° C.=30,000). In some embodiments, a=11.06; b=2.857; c=0.811; d=3.328 and e=2.966.

In some embodiments, lubricant base oil is used to formulate a high shear engine oil. In some embodiments, the stepwise regression utilizes at least three spectroscopy peak values. In some embodiments, the stepwise regression equation is a+b*P17+c*P18−d*P15+e*(P2+P4+P10)−f*(P1+P5)<LN (Cold Cranking Simulator Viscosity@−25° C.=7,000). In some embodiments, a=9.093; b=0.4957; c=2.842; d=1.850, e=2.094 and f′=1.964.

In some embodiments, the low temperature property is Mini Rotary Viscometer viscosity. In some embodiments, the stepwise regression utilizes at least three spectroscopy peak values. In some embodiments, the stepwise regression equation is a−b*P18+c*(P2+P4)<LN (Mini Rotary Viscosity@−30° C.=40,000). In some embodiments, a=12.18; b=4.16; and c=3.24.

In some embodiments, the set of samples span Group II, III and IV base oils.

In some embodiments, it may be desirable to use a different viscosity base oil. In such embodiments, the functional equations may be made relative to a well-known standard, such as API Group IV base stocks, especially 4, 6 and 8 cSt PAO.

In some embodiments, a ratio of other suitable techniques relative to the different viscosity can be used. For example, for any of the low temperature property predictions, an equation of the following form could be used: Predicted LTP Viscosity (Baseoil)<1.2*Predicted LTP Viscosity (PAO), where the viscosity of the PAO is the appropriate viscosity of reference.

In some embodiments, the reference viscosity range of the PAO may extend from 2 to 150 cSt @ 100° C.

In some embodiments, the form of the equation may be more complex to comprehend expected non-linearities. An example may be: Predicted LTP Viscosity (Baseoil)<1.2*F(29 cSt/kV40)*Predicted LTP Viscosity (PAO), where F(argument) is a function that could be a linear form, or could be exponential, logarithmic or a power law.

### EXAMPLES

Samples were prepared 25-30 wt % in CDCl3 with 7% Chromium (III)-acetylacetonate added as a relaxation agent. 13C NMR experiments were performed on a JEOL ECS NMR spectrometer, for which the proton resonance frequency is 400 MHz, in accordance with Quantitative 13C NMR Experiments were performed at 27° C. using an inverse gated decoupling experiment with a 45° flip angle, 6.6 seconds between pulses, 64 K data points and 2400 scans. All spectra were referenced to TMS at 0 ppm. Spectra were processed with 0.2-1 Hz of line broadening and baseline correction was applied prior to manual integration. Peaks are integrated as shown in FIG. 1 and Table 1, above. A macro was employed using NMR software from Advanced Chemistry Development Inc. (ACD Labs), of Toronto, Ontario, Canada, to assure that spectra were consistently integrated the same way.

Further illustrative, non-exclusive examples of systems and methods according to the present disclosure are presented in the following enumerated paragraphs. It is within the scope of the present disclosure that an individual step of a method recited herein, including in the following enumerated paragraphs, may additionally or alternatively be referred to as a “step for” performing the recited action.

### PCT/EP Clauses:

1. A lubricant base oil, the lubricant base oil having a low temperature property determined using a stepwise regression of carbon-13 nuclear magnetic resonance (NMR) spectroscopy peak values.

2. The lubricant base oil of clause 1, wherein the spectroscopy peak values used in the stepwise regression are significant at the 90 percent confidence level.

3. The lubricant base oil of clause 1 or 2, wherein the spectroscopy peak values used in the stepwise regression are significant at the 95 percent confidence level.

4. The lubricant base oil of clauses 1-3, wherein the lubricant base oil is a component of an industrial oil.

5. The lubricant base oil of clause 4, wherein the stepwise regression utilizes at least three spectroscopy peak values.

6. The lubricant base oil of clause 5, wherein the stepwise regression equation is a b*P15+c*P17−d*P18+e*(P2+P4+P10)<LN (Scanning Brookfield Viscosity@−30° C.=30,000).

7. The lubricant base oil of clause 6, wherein a=11.06; b=2.857; c=0.811; d=3.328 and e=2.966.

8. The lubricant base oil of clauses 1-3, wherein the lubricant base oil is a component of a high shear engine oil.

9. The lubricant base oil of clause 8, wherein the stepwise regression utilizes at least three spectroscopy peak values.

10. The lubricant base oil of clause 9, wherein the stepwise regression equation is a+b*P17+c*P118−d*P15+e*(P2+P4+P10)−f*(P1+P5)<LN (Cold Cranking Simulator Viscosity@−25 C=7,000).

11. The lubricant base oil of clause 10, wherein a=9.093; b=0.4957; c=2.842; d=1.850, e=2.094 and f=1.964.

12. The lubricant base oil of clauses 1-3, wherein the low temperature property is determined by Mini Rotary Viscometer viscosity (ASTM D4684).

13. The lubricant base oil of clause 12, wherein the stepwise regression utilizes at least three spectroscopy peak values.

14. The lubricant base oil of clause 13, wherein the stepwise regression equation is a−b*P18+c*(P2+P4)<LN (Mini Rotary Viscosity@−30° C.=40,000).

15. A method of selecting candidate lubricant base oils, or mixtures thereof, having acceptable low temperature performance, the method comprising: evaluating a set of samples using carbon-13 nuclear magnetic resonance (NMR) spectroscopy, each of the samples having a low temperature property; performing a stepwise regression of the carbon-13 NMR spectroscopy peak values obtained for the set of samples and their low temperature properties; selecting the carbon-13 NMR spectroscopy peak values found to be significant at the at least the 90% confidence level for the selected low temperature property; and selecting a candidate lubricant base oil based upon the regression equation.

16. An online method of blending a lubricant base oil, the method comprising: evaluating a set of samples using carbon-13 nuclear magnetic resonance (NMR) spectroscopy, each of the samples having a low temperature property; performing a stepwise regression of the carbon-13 NMR spectroscopy peak values obtained for the set of samples and their low temperature properties; selecting the carbon-13 NMR spectroscopy peak values found to be significant at the at least the 90% confidence level for the selected low temperature property; monitoring online the carbon-13 NMR spectroscopy peak values of a first lubricant base oil blending component; monitoring online the carbon-13 NMR spectroscopy peak values of at least a second lubricant base oil blending component; mathematically determining the optimal blend ratio of the first lubricant base oil blending component and the at least second lubricant base oil blending component; and blending the first lubricant base oil blending component and the at least second lubricant base oil blending component in accordance with the optimal blend ratio to form a lubricant base oil.

### INDUSTRIAL APPLICABILITY

The compositions and methods disclosed herein are applicable to the oil industry.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A finished lubricant comprising: a lubricant base oil having a low temperature property determined using a data analytics/machine learning technique on carbon-13 nuclear magnetic resonance (NMR) spectroscopy peak values.

2. The finished lubricant of claim 1, wherein the data analytics/machine learning technique comprises stepwise regression, Bayesian regression, LASSO/Ridge regression, random forest, support vector machine, or deep learning techniques.

3. The finished lubricant of claim 2, wherein the data analytics/machine learning technique comprises stepwise regression and the spectroscopy peak values used in the stepwise regression are significant at the 90 percent confidence level.

4. The finished lubricant of claim 3, wherein the spectroscopy peak values used in the stepwise regression are significant at the 95 percent confidence level.

5. The finished lubricant of claim 4, wherein the finished lubricant is an industrial oil.

6. The finished lubricant of claim 5, wherein the stepwise regression utilizes at least three spectroscopy peak values.

7. The finished lubricant of claim 6, wherein the stepwise regression equation is a−b*P15+c*P17−d*P18+e*(P2+P4+P10)<LN (Scanning Brookfield Viscosity@−30° C.=30,000).

8. The finished lubricant of claim 7, wherein a=11.06; b=2.857; c=0.811; d=3.328 and e=2.966.

9. The finished lubricant of claim 4, wherein the finished lubricant is a formulated engine oil for operation at high shear conditions.

10. The finished lubricant of claim 9, wherein the stepwise regression utilizes at least three spectroscopy peak values.

11. The finished lubricant of claim 10, wherein the stepwise regression equation is a+b*P17+c*P118−d*P15+e*(P2+P4+P10)−f*(P1+P5)<LN (Cold Cranking Simulator Viscosity@−25° C.=7,000).

12. The finished lubricant of claim 11, wherein a=9.093; b=0.4957; c=2.842; d=1.850, e=2.094 and f=1.964.

13. The finished lubricant of claim 4, wherein the low temperature property is viscosity as determined by Mini Rotary Viscometer (ASTM D4684).

14. The finished lubricant of claim 13, wherein the stepwise regression utilizes at least three spectroscopy peak values.

15. The finished lubricant of claim 14, wherein the stepwise regression equation is a−b*P18+c*(P2+P4)<LN (Mini Rotary Viscosity@−30° C.=40,000).

16. The finished lubricant of claim 15, wherein a=12.18; b=4.16; and c=3.24.

17. A method of selecting candidate lubricant base oils, or mixtures thereof, having acceptable low temperature performance, the method comprising:
evaluating a set of samples using carbon-13 nuclear magnetic resonance (NMR) spectroscopy, each of the samples having a low temperature property;
performing a data analytics/machine learning technique on the carbon-13 NMR spectroscopy peak values obtained for the set of samples and their low temperature properties;
selecting the carbon-13 NMR spectroscopy peak values found to be significant at the at least the 90% confidence level for the selected low temperature property; and
selecting a candidate lubricant base oil based upon the data analytics/machine learning technique.

18. The method of claim 17, wherein the data analytics/machine learning technique comprises stepwise regression, Bayesian regression, LASSO/Ridge regression, random forest, support vector machine, or deep learning techniques.

19. The method of claim 18, wherein the data analytics/machine learning technique comprises stepwise regression, and the spectroscopy peak values used in the stepwise regression are significant at the 95 percent confidence level.

20. The method of claim 19, wherein the lubricant base oil is used to formulate an industrial oil.

21. The method of claim 20, wherein the stepwise regression utilizes at least three spectroscopy peak values.

22. The method of claim 21, wherein the stepwise regression equation is a−b*P15+c*P17−d*P18+e*(P2+P4+P10)<LN (Scanning Brookfield Viscosity@−30° C.=30,000).

23. The method of claim 22, wherein a=11.06; b=2.857; c=0.811; d=3.328 and e=2.966.

24. The method of claim 19, wherein the lubricant base oil is used to formulate an engine oil for operation at high shear conditions.

25. The method of claim 24, wherein the stepwise regression utilizes at least three spectroscopy peak values.

26. The method of claim 25, wherein the stepwise regression equation is a+b*P17+c*P18−d*P15+e*(P2+P4+P10)−f*(P1+P5)<LN (Cold Cranking Simulator Viscosity@−25° C.=7,000).

27. The method of claim 26, wherein a=9.093; b=0.4957; c=2.842; d=1.850, e=2.094 and f=1.964.

28. The method of claim 19, wherein the low temperature property is Mini Rotary Viscometer viscosity (ASTM D4684).

29. The method of claim 28, wherein the stepwise regression utilizes at least three spectroscopy peak values.

30. The method of claim 29, wherein the stepwise regression equation is a−b*P18+c*(P2+P4)<LN (Mini Rotary Viscosity@−30° C.=40,000).

31. The method of claim 30, wherein a=12.18; b=4.16; and c=3.24.

32. The method of claim 17, wherein the set of samples span Group II, III and IV base oils.

33. A lubricant base oil, the lubricant base oil having a low temperature property determined using a data analytics/machine learning technique on carbon-13 nuclear magnetic resonance (NMR) spectroscopy peak values.

34. The lubricant base oil of claim 33, wherein the data analytics/machine learning technique comprises stepwise regression, Bayesian regression, LASSO/Ridge regression, random forest, support vector machine or deep learning techniques.

35. The lubricant base oil of claim 34, wherein the data analytics/machine learning technique comprises stepwise regression and the spectroscopy peak values used in the stepwise regression are significant at the 90 percent confidence level.

36. The lubricant base oil of claim 35, wherein the spectroscopy peak values used in the stepwise regression are significant at the 95 percent confidence level.

37. The lubricant base oil of claim 36, wherein the lubricant base oil is a component of an industrial oil.

38. The lubricant base oil of claim 37, wherein the stepwise regression utilizes at least three spectroscopy peak values.

39. The lubricant base oil of claim 38, wherein the stepwise regression equation is a−b*P15+c*P17−d*P18+e*(P2+P4+P10)<LN (Scanning Brookfield Viscosity@−30° C.=30,000).

40. The lubricant base oil of claim 39, wherein a=11.06; b=2.857; c=0.811; d=3.328 and e=2.966.

41. The lubricant base oil of claim 36, wherein the finished lubricant is a high shear engine oil.

42. The lubricant base oil of claim 41, wherein the stepwise regression utilizes at least three spectroscopy peak values.

43. The lubricant base oil of claim 42, wherein the stepwise regression equation is a+b*P17+c*P118−d*P15+e*(P2+P4+P10)−f*(P1+P5)<LN (Cold Cranking Simulator Viscosity@−25° C.=7,000).

44. The lubricant base oil of claim 43, wherein a=9.093; b=0.4957; c=2.842; d=1.850, e=2.094 and f=1.964.

45. The lubricant base oil of claim 36, wherein the low temperature property is Mini Rotary Viscometer viscosity (ASTM D4684).

46. The lubricant base oil of claim 45, wherein the stepwise regression utilizes at least three spectroscopy peak values.

47. The lubricant base oil of claim 46, wherein the stepwise regression equation is a−b*P18+c*(P2+P4)<LN (Mini Rotary Viscosity@−30° C.=40,000).

48. The lubricant base oil of claim 47, wherein a=12.18; b=4.16; and c=3.24.

49. The lubricant base oil of claim 33, wherein the predicted low temperature viscosity of the base oil <1.2*Predicted low temperature viscosity of a PAO, wherein the viscosity of the PAO is the viscosity of a reference oil.

50. The lubricant base oil of claim 49, wherein the reference viscosity range of the PAO is from 2 to 150 cSt @ 100° C.

51. The lubricant base oil of claim 33, wherein the predicted low temperature viscosity of the base oil <1.2*F(29 cSt/kV40)*Predicted low temperature viscosity of a PAO.

52. The lubricant base oil of claim 51, wherein the F (argument) is a linear form, an exponential form, a logarithmic form, or a power law form.

53. An online method of blending a lubricant base oil, the method comprising:
evaluating a set of samples using carbon-13 nuclear magnetic resonance (NMR) spectroscopy, each of the samples having a low temperature property;
performing a data analytics/machine learning technique on the carbon-13 NMR spectroscopy peak values obtained for the set of samples and their low temperature properties;
selecting the carbon-13 NMR spectroscopy peak values found to be significant at the at least the 90% confidence level for the selected low temperature property;
monitoring online the carbon-13 NMR spectroscopy peak values of a first lubricant base oil blending component;
monitoring online the carbon-13 NMR spectroscopy peak values of at least a second lubricant base oil blending component;
mathematically determining the optimal blend ratio of the first lubricant base oil blending component and the at least second lubricant base oil blending component; and
blending the first lubricant base oil blending component and the at least second lubricant base oil blending component in accordance with the optimal blend ratio to form a lubricant base oil.

54. The method of claim 53, wherein the data analytics/machine learning technique comprises stepwise regression, Bayesian regression, LASSO/Ridge regression, random forest, support vector machine or deep learning techniques.

55. The method of claim 54, wherein the data analytics/machine learning technique comprises stepwise regression and the spectroscopy peak values used in the stepwise regression are significant at the 90 percent confidence level.

56. The method of claim 55, wherein the spectroscopy peak values used in the stepwise regression are significant at the 95 percent confidence level.

57. The method of claim 56, wherein the lubricant base oil is used to formulate an industrial oil.

58. The method of claim 57, wherein the stepwise regression utilizes at least three spectroscopy peak values.

59. The method of claim 58, wherein the stepwise regression equation is a−b*P15+c*P17−d*P18+e*(P2+P4+P10)<LN (Scanning Brookfield Viscosity@−30° C.=30,000).

60. The method of claim 59, wherein a=11.06; b=2.857; c=0.811; d=3.328 and e=2.966.

61. The method of claim 60, wherein the lubricant base oil is used to formulate a high shear engine oil.

62. The method of claim 61, wherein the stepwise regression utilizes at least three spectroscopy peak values.

63. The method of claim 62, wherein the stepwise regression equation is a+b*P17+c*P18−d*P15+e*(P2+P4+P10)−f*(P1+P5)<LN (Cold Cranking Simulator Viscosity@−25° C.=7,000).

64. The method of claim 63, wherein a=9.093; b=0.4957; c=2.842; d=1.850, e=2.094 and f=1.964.

65. The method of claim 56, wherein the low temperature property is Mini Rotary Viscometer viscosity (ASTM D4684).

66. The method of claim 65, wherein the stepwise regression utilizes at least three spectroscopy peak values.

67. The method of claim 66, wherein the stepwise regression equation is a−b*P18+c*(P2+P4)<LN (Mini Rotary Viscosity@−30° C.=40,000).

68. The method of claim 67, wherein a=12.18; b=4.16; and c=3.24.

69. The method of claim 55, wherein the set of samples span Group II, III and IV base oils.
